(19) United States
(12) Patent Application Publication
(10) Pub. No.: US 2026/0294841 A1
(43) Pub. Date: Oct. 1, 2026

(54) GINGEROL FORMULATION AND METHODS FOR TREATING CANCER AND BACTERIAL INFECTION

(71) Applicant: UNIVERSITY OF TABUK, Tabuk (SA)

(72) Inventors: Mahmoud Ali ABDELAZIZ, Tabuk (SA); Adel Ibrahim Ahmad ALALAWY, Tabuk (SA); Mohamed Sobhi MOTAWEA, Tabuk (SA); Omar Mutlaq ALATAWI, Tabuk (SA); Omaymah Abdulaal ALAYSUY, Tabuk (SA)

(73) Assignee: UNIVERSITY OF TABUK, Tabuk (SA)

(21) Appl. No.: 19/090,097

(22) Filed: Mar. 25, 2025

(51) Int. Cl. A61K 31/12; A61K 9/51; A61K 36/9068; A61P 31/04; A61P 35/00

(52) U.S. Cl. CPC A61K 31/12; A61K 9/5123; A61K 9/5161; A61K 36/9068; A61P 31/04; A61P 35/00

(57) ABSTRACT

A method of reducing cell viability of colon cancer cells includes contacting an encapsulated gingerol composition with the colon cancer cells at a concentration of 15 to 100 μg/mL, wherein the encapsulated gingerol composition comprises gingerol nanoparticles extracted from Zingiber officinale, an ionic cross-linker, a polysorbate, and chitosan nanoparticles, wherein the ionic cross-linker is bonded to the chitosan. The encapsulated gingerol composition is a powder comprising nanoparticles having an average particle size of less than 100 nm, and contacting the encapsulated gingerol composition with the colon cancer cells reduces the cell viability of the colon cancer cells to 40% or less. A method of treating a bacterial infection with the encapsulated gingerol composition.

### BACKGROUND

### Technical Field

The present disclosure is directed to nanoparticle formulation, more particularly, towards a gingerol encapsulated nanoparticle formulation for treatment against colon cancer cells and bacterial infection.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

Colon cancer and bacterial infections are among the leading causes of death and disability on a worldwide scale. Colon cancer is a type of cancer that begins in the colon (large intestine) or rectum. It often starts as small, noncancerous growths called polyps that may become cancerous over time. Bacterial infections are caused by harmful bacteria that enter the body and multiply, leading to a variety of health problems. Such infections may affect different parts of the body, ranging from mild to severe.

Chemotherapy for cancer and antibiotics for bacterial infections are two examples of conventional therapies with significant limits. As a consequence of its non-specific effects on both malignant and healthy cells, chemotherapy may reduce tumor development but comes with serious side effects such as nausea, exhaustion, and immunological suppression. Colon cancer is the most destructive form of the disease, destroying an estimated 10% of those diagnosed with cancer globally. Treatment typically involves surgery to remove the tumor, chemotherapy, radiation therapy, and sometimes targeted therapy or immunotherapy, depending on the stage and specific characteristics of the cancer. The two most common forms of treatment today, chemotherapy and surgery, are costly and have horrible side effects. Chemotherapy for colon cancer may cause various side effects, including fatigue, nausea, vomiting, hair loss, diarrhea, and a weakened immune system, leading to increased risk of infections. Other potential effects include mouth sores, changes in appetite, and neuropathy (nerve damage), which may vary based on the specific drugs used. Despite its widespread benefit, chemotherapy is not always selective and may kill both cancerous and noncancerous cells. Colon cancer chemotherapy presents a number of bioavailability issues as well. Especially in cases that are advanced or metastatic, drugs frequently spread systemically, limiting their concentration at the tumor site. The liver may first-pass metabolize oral chemotherapy medications, lowering their bloodstream availability. Effectiveness is further compromised by poor medication delivery to deep or dense tumors and limited tumor penetration. Furthermore, resistance mechanisms such efflux pumps, which lower medication concentrations at the target location, may be developed by cancer cells. Together, these problems reduce chemotherapy's therapeutic efficacy while raising side effects because of the non-specific medication distribution.

Bacterial infections are typically treated with antibiotics, which target and kill the bacteria or inhibit its growth. However, antibiotic resistance is a growing concern, so it's important to take antibiotics only as prescribed by a healthcare provider. In the medical sector, advancing pathogens resistant to antibiotics is a further significant issue. Antimicrobial resistance is anticipated to outperform cancer as the primary cause of global mortality, resulting in 10 million fatalities annually. Bacterial infections are treated with antibiotics like penicillin, amoxicillin, ciprofloxacin, or doxycycline. While effective, antibiotics may cause side effects such as nausea, diarrhea, rashes, or allergic reactions. Overuse may lead to antibiotic resistance, reducing treatment efficacy. Fluoroquinolones may cause tendon damage, while macrolides may cause liver issues. Topical antibiotics like neomycin may cause skin irritation. IV antibiotics, though necessary for severe infections, may lead to infections at the injection site, kidney damage, or blood clots. Overuse and abuse of traditional antibiotics may cause bacterial infections to develop resistance, which is linked to longer hospital stays, higher medical costs, and higher death rates. Additionally, low water solubility of antibiotics may pose as an obstacle for their absorption and bioavailability, reducing their effectiveness against bacterial infections. Poor aqueous solubility may limit drug concentration in the bloodstream and tissues, especially in target areas, leading to suboptimal therapeutic outcomes. This may lead to higher doses or alternative delivery methods.

Each of the aforementioned treatment methods suffers from one or more drawbacks hindering their adoption. Accordingly, it is one object of the present disclosure to provide a treatment method that may circumvent problems like lack of bioavailability, quick breakdown in the body, low water solubility, high cost, and non-specificity towards targeted cells, etc. known in the art.

### SUMMARY

In an exemplary embodiment, a method of reducing cell viability of colon cancer cells is described. The method includes contacting an encapsulated gingerol composition with the colon cancer cells at a concentration of 15 μg/mL to 100 μg/mL, where the encapsulated gingerol composition includes gingerol nanoparticles extracted from Zingiber officinale, an ionic cross-linker, a polysorbate, and chitosan nanoparticles. The ionic cross-linker is bonded to the chitosan. The encapsulated gingerol composition is a powder including nanoparticles having an average particle size of less than 100 nm. Contacting the encapsulated gingerol composition with the colon cancer cells reduces the cell viability of the colon cancer cells to 40% or less.

In some embodiments, the colon cancer cells are HT-29 and contacting the encapsulated gingerol composition with the colon cancer cells at a concentration of 25 μg/mL reduces the colon cancer cell viability to 35% or less.

In some embodiments, the colon cancer is HCT116 and the contacting the encapsulated gingerol composition with the colon cancer cells at a concentration of 15 μg/mL reduces the colon cancer cell viability to 30% or less.

In some embodiments, the ionic cross-linker is at least one selected from the group consisting of sodium tri metaphosphate (STMP), glutaraldehyde, genipin, epichlorohydrin, phosphoryl chloride, N,N′-methylenebisacrylamide, citric acid, adipic acid, starch dialdehyde, and sodium tripolyphosphate.

In some embodiments, the polysorbate is at least one selected from the group consisting of polysorbate 20, polysorbate 40, polysorbate 60, and polysorbate 80.

In some embodiments, the method of contacting the encapsulated gingerol composition with the colon cancer cells includes contacting the encapsulated gingerol composition with the colon cancer cells at a concentration of 15 μg/mL to 50 μg/mL.

In some embodiments, the polysorbate is polysorbate 80.

In some embodiments, the ionic cross-linker is sodium tripolyphosphate.

In another exemplary embodiment, a method of treating a bacterial infection is described. The method includes contacting bacteria of the bacterial infection with an encapsulated gingerol composition at a concentration of 15 μg/mL to 100 μg/mL to kill the bacteria. The encapsulated gingerol composition includes gingerol nanoparticles extracted from Zingiber officinale, an ionic crosslinker, a polysorbate, and chitosan nanoparticles, where the ionic cross-linker is bonded to the chitosan. The encapsulated gingerol composition is a powder including nanoparticles having an average particle size of less than 100 nm, and where the bacterial infection includes at least one bacterium selected from the group including of Staphylococcus aureus, K. pneumoniae, E. faecalis, P. aeruginosa, E. coli, and A. baumannii.

In some embodiments, the encapsulated gingerol composition with the bacteria has a zone of inhibition of at least 20 mm against a Staphylococcus aureus bacterium.

In some embodiments, contacting the encapsulated gingerol composition with the bacteria has a zone of inhibition of at least 18 mm against a Klebsiella pneumoniae bacterium.

In some embodiments, contacting the encapsulated gingerol composition with the bacteria has a zone of inhibition of at least 17 mm against an Enterococcus faecalis bacterium.

In some embodiments, contacting the encapsulated gingerol composition with the bacteria has a zone of inhibition of at least 13 mm against a Pseudomonas aeruginosa bacterium.

In some embodiments, contacting the encapsulated gingerol composition with the bacteria has a zone of inhibition of at least 16 mm against an Escherichia coli bacterium.

In some embodiments, contacting the encapsulated gingerol composition with the bacteria has a zone of inhibition of at least 14 mm against an Acinetobacter baumannii bacterium.

In some embodiments, the ionic cross-linker is at least one selected from the group consisting of sodium tri metaphosphate (STMP), glutaraldehyde, genipin, epichlorohydrin, phosphoryl chloride, N,N′-methylenebisacrylamide, citric acid, adipic acid, starch dialdehyde, and sodium tripolyphosphate.

In some embodiments, the polysorbate is at least one selected from the group consisting of polysorbate 20, polysorbate 40, polysorbate 60, and polysorbate 80.

In some embodiments, the ionic cross-linker is sodium tripolyphosphate.

In some embodiments, the polysorbate is polysorbate 80.

In some embodiments, the contacting includes contacting the encapsulated gingerol composition with bacteria at a concentration of 15 μg/mL to 50 μg/mL.

The foregoing general description of the illustrative embodiments and the following detailed description thereof are merely exemplary aspects of the teachings of this disclosure, and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIGS. 1A-1D show transmission electron microscopy (TEM) images of a gingerol compound (GC) encapsulated chitosan nanocomposite (GC@CsNC) at different magnifications and different positions, according to certain embodiments.

FIGS. 2A-2F shows scanning electron microscopy (SEM) images of the GC@CsNC at different magnifications and different positions, according to certain embodiments.

FIG. 3 shows cytotoxicity and cell viability of colon cancer cells treated with GC@CsNC using microculture tetrazolium test (MTT) assay, according to certain embodiments.

FIG. 4 shows kinetics release of gingerol from the GC@CsNC, according to certain embodiments.

FIG. 5 shows an antibacterial activated and zone of inhibition (ZOI) in mm of GC@CsNC against various pathogenic bacteria, according to certain embodiments.

### DETAILED DESCRIPTION

In the drawings, like reference numerals designate identical or corresponding parts throughout the several views. Further, as used herein, the words “a”, “an” and the like generally carry a meaning of “one or more”, unless stated otherwise.

Furthermore, the terms “approximately,” “approximate”, “about” and similar terms generally refer to ranges that include the identified value within a margin of 20%, 10%, or preferably 5%, and any values therebetween.

When describing the present disclosure, the terms used are to be construed in accordance with the following definitions, unless a context dictates otherwise.

Embodiments of the present invention will now be described more fully hereinafter with reference to the accompanying drawings wherever applicable, in that some, but not all, embodiments of the disclosure are shown.

In the drawings, like reference numerals designate identical or corresponding parts throughout the several views. Further, as used herein, the words ‘a,’ ‘an’ and the like generally carry a meaning of ‘one or more,’ unless stated otherwise.

Furthermore, the terms ‘approximately,’ ‘approximate,’ ‘about,’ and similar terms generally refer to ranges that include the identified value within a margin of 20%, 10%, or preferably 5%, and any values therebetween.

As used herein, the term ‘cell viability’ refers to the proportion of live, healthy cells in a population after being exposed to various conditions or treatments. It is commonly measured to assess the health and functionality of cells in laboratory studies, particularly when testing the effects of drugs, toxins, or other experimental variables. Viable cells can maintain their metabolism, growth, and division processes, while non-viable cells are damaged, dysfunctional, or dead. Cell viability is often assessed using assays that measure metabolic activity, membrane integrity, or other indicators of cellular health, e.g., luminescent cell viability, ATP cell viability, tetrazolium reduction cell viability, live-cell protease cell viability, DNA synthesis proliferation, metabolic proliferation cell viability, fluorescent dye proliferation and luminescent ATP cell viability.

As used herein, the term ‘ionic cross-linker’ is a chemical agent that facilitates the formation of cross-links between molecules or polymer chains through ionic bonds. These cross-links are formed by the interaction of positively charged ions (cations) with negatively charged groups (anions) on the molecules or polymers, leading to a network structure.

As used herein, the term ‘cancer’ refers to all types of cancer, neoplasm or malignant tumors found in mammals (e.g. humans), including leukemias, lymphomas, carcinomas, and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

As used herein, the terms ‘treat,’ ‘treatment,’ and ‘treating’ in the context of the administration of a therapy to a subject in need thereof refer to the reduction or inhibition of the progression and or duration of cancer, the reduction or amelioration of the severity of cancer, and/or the amelioration of one or more symptoms thereof resulting from the administration of one or more therapies. In some embodiments, the subject is a mammalian subject. In one embodiment, the subject is a human. ‘Treating’ or ‘treatment’ of a disease includes preventing the disease from occurring in a subject that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease). With regard to cancer or hyperplasia, these terms simply mean that the life expectancy of an individual affected with cancer will be increased or that one or more of the symptoms of the disease will be reduced. In specific embodiments, such terms refer to one, two or three or more results following the administration of one, two, three or more therapies: (1) a stabilization, reduction or elimination of the cancer stem cell population; (2) a stabilization, reduction or elimination in the cancer cell population; (3) a stabilization or reduction in the growth of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate, (10) a decrease in hospitalization lengths, (11) the size of the tumor is maintained and does not increase or increases by less than 10%, preferably less than 5%, preferably less than 4%, preferably less than 2%, and (12) an increase in the number of patients in remission. In certain embodiments, such terms refer to a stabilization or reduction in cancer stem cell population. In some embodiments, such terms refer to a stabilization or reduction in the growth of cancer cells. In some embodiments, such terms refer to stabilization or reduction in cancer stem cell population and a reduction in the cancer cell population. In some embodiments, such terms refer to a stabilization or reduction in the growth and or formation of a tumor. In some embodiments, such terms refer to the eradication, removal, or control of primary, regional, or metastatic cancer (e.g., the minimization or delay of the spread of cancer). In some embodiments, such terms refer to a reduction in mortality and/or an increase in the survival rate of a patient population. In further embodiments, such terms refer to an increase in the response rate, the durability of response, or the number of patients who respond or are in remission. In some embodiments, such terms refer to a decrease in the hospitalization rate of a patient population and/or a decrease in hospitalization length for a patient population.

As used herein, the term ‘pharmaceutical composition’ refers to a mixture of the compounds described herein or pharmaceutically acceptable salts, esters, or prodrugs thereof, with other chemical components, such as physiologically acceptable carriers and excipients.

As used herein, the term ‘excipient’ refers to an inert substance added to a pharmaceutical composition to facilitate the administration of a compound further. Examples, without limitation, of excipients include calcium carbonate, calcium phosphate, various sugars and types of starch, cellulose derivatives, gelatin, vegetable oils, and polyethylene glycols (PEGs).

As used herein, the term ‘administering’ means oral administration, administration as a suppository, topical contact, intravenous, parenteral, intraperitoneal, intramuscular, intralesional, intrathecal, intranasal, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent. In some embodiments, the administration of the anticancer compound is selected from a group including intravenous, interperitoneally, intramuscular, and oral administration.

As used herein, the term ‘nanoparticles (NPs)’ refer to the small particles that have at least one dimension in the range of 1 to 100 nm. Their size, shape, and surface properties can be manipulated at the molecular or atomic level. Due to their small size, NPs exhibit unique physical and chemical properties compared to their bulk counterparts, such as increased surface area, enhanced reactivity.

As used herein, the term ‘primary emulsifier’ refers to a substance that plays a key role in stabilizing an emulsion, which is a mixture of two immiscible liquids (such as oil and water). The primary emulsifier reduces the surface tension between the liquids, allowing them to form a stable mixture by coating the droplets of one liquid and preventing them from coalescing. Primary emulsifiers are typically surfactants or amphiphilic molecules that possess both hydrophilic (water-attracting) and hydrophobic (oil-attracting) properties. Common examples of primary emulsifiers include lecithin, sodium stearoyl lactylate, polysorbates, and monoglycerides.

As used herein, the term ‘zone of inhibition’ refers to the area around an antimicrobial agent (such as an antibiotic or antiseptic) on an agar plate where bacterial growth is inhibited. It is observed as a clear, circular region surrounding the disc or well containing the antimicrobial substance. The size of the zone of inhibition is measured in millimetres and indicates the effectiveness of the antimicrobial agent in preventing bacterial growth. A larger zone typically suggests a stronger antimicrobial effect, while a smaller zone indicates less efficacy. The zone of inhibition is commonly used to evaluate the antibacterial activity of a substance.

The present disclosure is intended to include all hydration states of a given compound or formula, unless otherwise noted or when heating a material.

Aspects of the present disclosure are directed to a sustainable method for administering medication targeting colon cancer and bacterial infections by encapsulating gingerol in biodegradable chitosan NPs. The formulation improves gingerol's stability, bioavailability, and therapeutic efficacy through controlled release and targeted distribution, while minimizing systemic toxicity. It demonstrates cytotoxicity against colon cancer cells and effectively inhibits the growth of bacterial pathogens, including Klebsiella pneumoniae and Staphylococcus aureus. The present disclosure addresses issues of medication resistance and environmental harm by utilizing green chemistry and renewable materials, offering potential applications in cancer, antimicrobial treatments, and nutraceuticals.

A method of reducing cell viability of colon cancer cells is described. The method includes contacting an encapsulated gingerol composition with the colon cancer cells at a concentration of 15-100 μg/mL, preferably 20-95 μg/mL, preferably 25-90 μg/mL, preferably 30-85 μg/mL, preferably 35-80 μg/mL, preferably 40-75 μg/mL, preferably 45-70 μg/mL, preferably 50-65 μg/mL, and preferably 55-60 μg/mL. In some embodiments, the encapsulated gingerol composition is contacted with the colon cancer cells at a concentration of 15-50 μg/mL, preferably 16-49 μg/mL, preferably 17-48 μg/mL, preferably 18-47 μg/mL, preferably 19-46 μg/mL, preferably 20-45 μg/mL, preferably 21-44 μg/mL, preferably 22-43 μg/mL, preferably 23-42 μg/mL, preferably 24-41 μg/mL, preferably 25-40 μg/mL, preferably 26-39 μg/mL, preferably 27-38 μg/mL, preferably 28-37 μg/mL, preferably 29-36 μg/mL, preferably 30-35 μg/mL, preferably 31-34 μg/mL, and preferably 32-33 μg/mL. In one aspect of the invention contacting the gingerol composition with cancer cells includes mixing or contacting a solution or suspension of the gingerol nanoparticles with the cancer cells. The cancer cells may be present in a subject or present in a container, for example, in vivo or in vitro. The microculture tetrazolium (MTT) assay was used to evaluate the cytotoxicity and cell viability of colon cancer cells (HT-29 and HCT-116) treated with the gingerol-encapsulated chitosan nanoparticles (GC@CsNC).

Gingerol is a phenolic molecule extracted from ginger (Zingiber officinale). It is one of many natural substances recently gaining attention as potential cancer treatments. Gingerol has strong anticancer effects, such as reducing tumor cell proliferation, increasing cell death, and preventing cancer from spreading. The encapsulated gingerol composition includes gingerol as the active ingredient, which is encapsulated within a protective carrier material. The carrier material may be selected from the group including lipids, polysaccharides, proteins, and synthetic polymers. The encapsulation enhances the stability, bioavailability, and controlled release of gingerol. The composition is designed to reduce degradation, improve solubility, and allow for a sustained or targeted release of gingerol in a subject. Additionally, the encapsulated gingerol composition minimizes direct irritation or bitterness associated with unencapsulated gingerol. As used herein, the subject in need thereof refers to a mammalian subject, preferably a human subject, who has been diagnosed with, is suspected of having, is susceptible to, is genetically predisposed to, or is at risk of having at least one form of cancer.

In some embodiments, gingerol may be encapsulated in lipophilic materials, such as lipids or phospholipids, to form lipid-based NPs for enhanced absorption. In some embodiments, polysaccharides like alginate or chitosan may be used as carriers, providing controlled release properties. In some embodiments, protein-based carriers such as gelatin or casein may be employed for sustained release and stability. In some embodiments, biodegradable polymers like poly(lactic-co-glycolic acid) (PLGA) or PEG encapsulate gingerol, allowing for precise control over release kinetics. In some embodiments, nanoencapsulation may be achieved using solid lipid NPs or polymeric micelles to improve solubility and bioavailability. In some embodiments, gingerol may be encapsulated in microspheres for gradual and controlled release.

The encapsulated gingerol composition includes gingerol NPs extracted from Zingiber officinale, an ionic cross-linker, a polysorbate, and chitosan NPs. In some embodiments, gingerol NPs are extracted from Zingiber officinale, primarily from the rhizome, which contains gingerol, shogaol, and zingerone. In some embodiments, shogaol, and zingerone NPs may also be used along with gingerol NPs. Other embodiments involve extracting gingerol NPs from the leaves or flowers, though in smaller amounts. Essential oils derived from fresh or dried rhizomes and dried ginger, or ginger powder can also be used.

In some embodiments, the polysorbate is at least one selected from the group including polysorbate 20, polysorbate 40, polysorbate 60, and polysorbate 80. In a preferred embodiment, the polysorbate is polysorbate 80. The polysorbate acts as a primary emulsifier. Polysorbate 80 helps to improve the solubility and uniform dispersion of ginger's active compounds in water-oil mixtures, ensuring stable emulsions and preventing phase separation. This enhances the bioavailability, effectiveness, and shelf-life of the encapsulated gingerol composition.

The ionic cross-linker is bonded to chitosan. Due to its favorable surface charge, the biodegradable and biocompatible biopolymer chitosan serves as an ideal carrier. It allows for targeted delivery to diseased tissues, such as colon cancer cells, while leaving healthy tissues unaffected. This process helps prevent the rapid degradation of gingerol and promotes its efficient cellular uptake, thus minimizing any potential side effects. Additionally, chitosan's inherent biodegradability reduces the risk of accumulation over time, as it breaks down into harmless metabolites. The ionic cross-linker bonded to the chitosan in the encapsulated gingerol composition enhances stability, ensures controlled release of gingerol, and improves its bioavailability by forming a protective network around the active compound. In some embodiments, the ionic cross-linker is at least one selected from the group including sodium tri metaphosphate (STMP), glutaraldehyde, genipin, epichlorohydrin, phosphoryl chloride, N,N′-methylenebisacrylamide, citric acid, adipic acid, starch dialdehyde, and sodium tripolyphosphate. Other examples of the ionic cross-linkers, in addition to mentioned, may include calcium chloride, zinc chloride, aluminum chloride, magnesium chloride, ferric chloride, borax (sodium tetraborate), potassium dichromate, iron sulfate, copper sulfate, and sodium sulfate. In a preferred embodiment, the ionic cross-linker is sodium tripolyphosphate.

The encapsulated gingerol composition is a powder including NPs having an average particle size of less than 100 nm, preferably 90 nm, preferably 85 nm, preferably 80 nm, preferably 75 nm, preferably 70 nm, preferably 65 nm, and preferably 60 nm. In some embodiments, the encapsulated gingerol composition may be provided in various forms, including liquid, gel, cream, or capsule, each offering specific advantages. The liquid form facilitates faster absorption and enhanced bioavailability, while gel or cream formulations are particularly suitable for targeted topical application. Capsules provide the advantage of controlled oral dosage. The powder form, particularly when incorporating NPs with an average particle size of less than 100 nm, provides significant benefits, including improved stability, extended shelf life, and increased bioavailability due to the greater surface area of the NPs.

In some embodiments, the contacting reduces the cell viability of the colon cancer cells to 40% or less, preferably 35%, preferably 30%, preferably 25%, preferably 20%. In some embodiments, the colon cancer cells are HT-29 and the contacting at a concentration of 25 μg/mL reduces the colon cancer cell viability to 35% or less, preferably 34%, preferably 33%, preferably 32%, preferably 31%, preferably 30%, preferably 29%, preferably 28%, preferably 27%, preferably 26%, preferably 25%, and preferably 24%. In a preferred embodiment, the colon cancer cells are HT-29 and the contacting at a concentration of 25 μg/mL reduces the colon cancer cell viability to 35%. HT-29 cells are a human colon cancer cell line derived from a patient with colorectal adenocarcinoma. In some embodiments, the colon cancer is HCT116 and the contacting at a concentration of 15 μg/mL reduces the colon cancer cell viability to 30% or less, preferably 29%, preferably 28%, preferably 27%, preferably 26%, preferably 25%, and preferably 24%. In a preferred embodiment, the colon cancer is HCT116 and the contacting at a concentration of 15 μg/mL reduces the colon cancer cell viability to preferably 25%. HCT116 cells are a human colon cancer cell line derived from a patient with metastatic colorectal carcinoma.

In another aspect, a method of treating a bacterial infection is described. The method includes contacting bacteria of the bacterial infection with an encapsulated gingerol composition at a concentration of 15-100 μg/mL, preferably 20-95 μg/mL, preferably 25-90 μg/mL, preferably 30-85 μg/mL, preferably 35-80 μg/mL, preferably 40-75 μg/mL, preferably 45-70 μg/mL, preferably 50-65 μg/mL, and preferably 55-60 μg/mL to kill the bacteria. In some embodiments, the contacting includes contacting the encapsulated gingerol composition with bacteria at a concentration of 15-50 μg/mL, preferably 16-49 μg/mL, preferably 17-48 μg/mL, preferably 18-47 μg/mL, preferably 19-46 μg/mL, preferably 20-45 μg/mL, preferably 21-44 μg/mL, preferably 22-43 μg/mL, preferably 23-42 μg/mL, preferably 24-41 μg/mL, preferably 25-40 μg/mL, preferably 26-39 μg/mL, preferably 27-38 μg/mL, preferably 28-37 μg/mL, preferably 29-36 μg/mL, preferably 30-35 μg/mL, preferably 31-34 μg/mL, and preferably 32-33 μg/mL.

In some embodiments, the encapsulated gingerol composition includes gingerol nanoparticles extracted from Zingiber officinale, an ionic crosslinker, a polysorbate, and chitosan nanoparticles, wherein the ionic cross-linker is bonded to the chitosan. In some embodiments, the ionic cross-linker is at least one selected from the group including STMP, glutaraldehyde, genipin, epichlorohydrin, phosphoryl chloride, N,N′-methylenebisacrylamide, citric acid, adipic acid, starch dialdehyde, and sodium tripolyphosphate. In a preferred embodiment, the ionic cross-linker is sodium tripolyphosphate.

In some embodiments, the encapsulated gingerol composition is a powder including nanoparticles having an average particle size of less than 100 nm, preferably 90 nm, preferably 85 nm, preferably 80 nm, preferably 75 nm, preferably 70 nm, preferably 65 nm, and preferably 60 nm.

In some embodiments, the polysorbate is at least one selected from the group including polysorbate 20, polysorbate 40, polysorbate 60, and polysorbate 80. In a preferred embodiment, the polysorbate is polysorbate 80.

In some embodiments, the bacterial infection includes at least one bacterium selected from the group including Staphylococcus aureus, Klebsiella pneumoniae, Enterococcus faecalis, P. aeruginosa, Escherichia coli, and Acinetobacter baumannii. In alternate embodiments, the bacterial infection may include bacterium, but is not limited to, Acinetobacter baumannii, Streptococcus pneumoniae, Streptococcus pyogenes, Enterobacter cloacae, Proteus mirabilis, Haemophilus influenzae, Salmonella enterica, Listeria monocytogenes, Clostridium difficile, Neisseria gonorrhoeae, Neisseria meningitidis, Bacillus anthracis, Campylobacter jejuni, Vibrio cholerae, Mycobacterium tuberculosis, Mycobacterium avium, Legionella pneumophila, Yersinia pestis, Francisella tularensis, Stenotrophomonas maltophilia, Moraxella catarrhalis, Corynebacterium diphtheriae, Bacteroides fragilis, Clostridium perfringens, Serratia marcescens, Citrobacter freundii, Providencia stuartii, Corynebacterium jeikeium, Bacillus cereus, Acinetobacter lwoffii, Pseudomonas fluorescens, Pseudomonas putida, Staphylococcus epidermidis, Streptococcus agalactiae, Enterococcus faecium, Klebsiella oxytoca, Helicobacter pylori, Eikenella corrodens, Pasteurella multocida, Bordetella pertussis, Chlamydia trachomatis, Chlamydia pneumoniae, Rickettsia rickettsii, Leptospira interrogans, and Treponema pallidum.

In some embodiments, contacting the encapsulated gingerol composition with the bacteria has a zone of inhibition of at least 20 mm, preferably 21 mm, and yet more preferably 22 mm against a Staphylococcus aureus bacterium. In some embodiments, contacting the encapsulated gingerol composition with the bacteria has a zone of inhibition of at least 18 mm, preferably 19 mm, preferably 20 mm, preferably 21 mm, and yet more preferably 22 mm against a Klebsiella pneumoniae bacterium. In some embodiments, contacting the encapsulated gingerol composition with the bacteria has a zone of inhibition of at least 17 mm, preferably 18 mm, and yet more preferably 19 mm against an Enterococcus faecalis bacterium. In some embodiments, contacting the encapsulated gingerol composition with the bacteria has a zone of inhibition of at least 13 mm, preferably 14 mm, and yet more preferably 15 mm against a Pseudomonas aeruginosa bacterium. In some embodiments, contacting the encapsulated gingerol composition with the bacteria has a zone of inhibition of at least 16 mm, preferably 17 mm, and yet more preferably 18 mm against an Escherichia coli bacterium. In some embodiments, contacting the encapsulated gingerol composition with the bacteria has a zone of inhibition of at least 14 mm, preferably 15 mm, and yet more preferably 16 mm against an Acinetobacter baumannii bacterium.

### Examples

The following examples demonstrate an encapsulated gingerol composition as described herein. The examples are provided solely for illustration and are not to be construed as limitations of the present disclosure, as many variations thereof are possible without departing from the spirit and scope of the present disclosure.

### Example 1: Isolation of Gingerol Compound from Crude Ginger Extract

Gingerol was extracted using a systematic approach. The ginger rhizomes were ground into a powder after being washed, peeled, and dried. The extraction solvent, ethanol, is an organic solvent inducted in the thimble of a Soxhlet device. The solvent was heated to vaporization and then condensed in the Soxhlet chamber, which percolated constantly over the ginger powder as part of the extraction process. The solvent usually took 6 hrs to 8 hrs to become transparent, indicating the extraction was complete. After passing the mixture through a filter to remove any particles, the solvent was diminished from the crude gingerol compound using a rotary evaporator [See: Said, P. P., O. P. Arya, R. C. Pradhan, R. S. Singh, and B. N. Rai (2015) Separation of oleoresin from ginger rhizome powder using green processing technologies, incorporated herein by reference in its entirety]. The original extract has received additional purification. After combining the gingerol fractions, the solvent was removed using thin layer chromatography (TLC) [See: Sarada, R., R. Vidhyavathi, D. Usha, and G. A. Ravishankar (2006) an efficient method for extraction of astaxanthin from green alga haematococcus pluvialis, incorporated herein by reference in its entirety]. Crystallization was used to purify the substance further, making it even more pure. High performance liquid chromatography (HPLC) and TLC were used to confirm that the extracted gingerol was of high quality. An improved gingerol compound suitable for scientific or medicinal applications was generated by the above method.

### Example 2: Preparation of Gingerol Compound (GC) Encapsulated Chitosan Nanocomposite

The gingerol compound (GC) was chilled for 6 hrs after it was collected. Subsequently, it should be exposed to a cold environment (−60° C.) in a freeze dryer for an entire day. Gingerol was reduced to a fine powder after the dehydrating procedure was finished. Solutions A and B were generated by dissolving 0.5 g of chitosan in 30 milliliter (mL) of acetic acid solution [0.5% volume/volume (V/V)] and agitating at ambient temperature for 12 hrs. 0.1 g of sodium tripolyphosphate (STPP) was agitated with 10 mL of deionised water in solution B for 15 minutes at ambient temperature. In the subsequent phase, 1 mL of Tween 80 was added to solution B, and the mixture was stirred continuously for an additional 10 minutes. Subsequently, 0.05 g of pulverised gingerol compound was added to solution B after 30 minutes of agitating. Solution B was added to solution A dropwise while agitating for 6 hrs after the extract had dissolved completely. The white solution was produced at the conclusion of the addition due to the ionic gelation process of STPP with chitosan. Finally, the solution was centrifuged at 10000 revolutions per minute (rpm) and dried using a freeze dryer machine at −60° C. The synthesized freeze-dried powder is a gingerol compound loaded with chitosan nanocomposite (GC@CsNC). The produced fine powder (GC@CsNC) was kept away from moisture to characterize using transmission electron microscopy (TEM) and scanning electron microscopy (SEM).

### Example 3: In Vitro Assessment Colon Cancer Therapy Using Microculture Tetrazolium Test (MTT) Assay

Colon cancer cell lines (HT-29 and HCT-116) were subjected to the MTT assay to determine the cytotoxicity of GC@CsNC. A 96-well plate was seeded with cells at a density of around 1×10⁴ cells/well and allowed to adhere for the night. Afterwards, for a duration of 24 hrs, the cells were subjected to varying concentrations of GC@CsNC (25 μg/mL, 50 μg/mL, and 100 μg/mL). The control group consisted of the cells that were not treated. After the first incubation, the medium was changed to one that included 5 mg/mL of MTT reagent, and the cells were left to incubate for another 4 hrs at 37° C. Using a microplate reader, the formazan crystals were dissolved in dimethyl sulfoxide (DMSO) and then the absorbance at 570 nanometer (nm) was measured.

### Example 4: Evaluation of Antibacterial Properties

The effectiveness of GC@CsNC as an antibacterial agent was evaluated by the well agar diffusion assay. Microbiological solutions were introduced to newly made Mueller-Hinton agar plates using a 0.5 McFarland standard for calibration. A specified concentration of GC@CsNC was added to 100 μL of agar after 6 mm diameter wells were constructed. After 24 hrs of incubation at 37° C., the zones of inhibition (ZOI) around the wells were assessed. In order to determine how efficient GC@CsNC was as an antibacterial agent against the tested bacteria, the data were recorded [See: Salem, S. S. (2022) Baker's Yeast-Mediated Silver Nanoparticles: Characterisation and antimicrobial biogenic tool for suppressing pathogenic microbes].

The use of GC is limited and restricted by some factors and environmental conditions such as fast release (uncontrolled) and its degradation at different pH. Therefore, the situation was circumvented, and its medical use was expanded by encapsulating it inside a biopolymer (chitosan), which is cheap and widely available in nature. The polymer greatly enhances its usability after the preparation in small size. The one pot synthesis of chitosan loaded with extract in the nanoform (GE@CsN) has many advantages such as the sustained release and its release at different pH. The materials in its nanoform enhance the delivery of the drug or extract directly to the injured or illness organ without its decomposition or dissolution. Before the medical utilization of the nanocomposite (GC@CsNC), its synthesis has been affirmed using TEM and SEM analysis.

By calculating the encapsulation efficiency of the GC inside chitosan nanoparticles, it was found that the encapsulation efficiency was 97.42%, which is considered a very large percent for the encapsulation and from which it is possible to expect the maximum benefit from the encapsulated GC. In addition, encapsulating it in a large proportion may be beneficial in treating cancer by the sustained release over a long time, which increases its effectiveness.

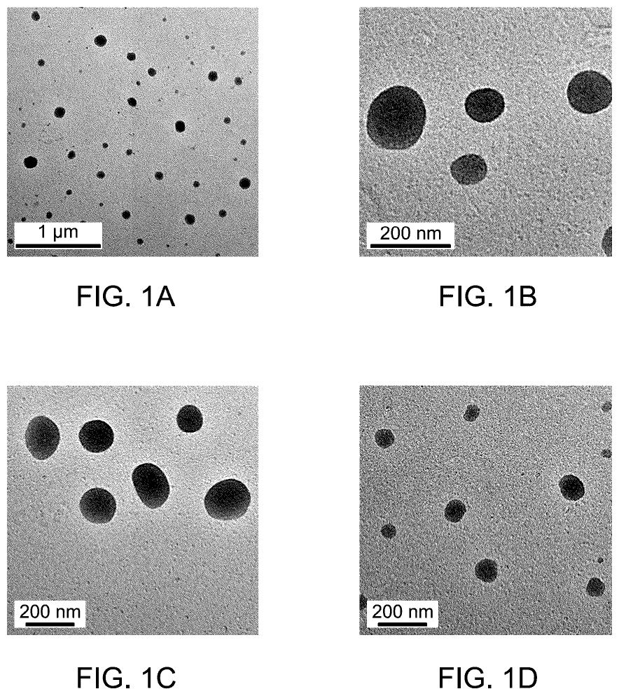

FIG. 1 depicts the TEM of GC@CsNC at different magnifications which proved the synthesis with nearly uniform particles and small size. In addition, FIG. 1 represents a well dispersed particles with no markable noticeable for agglomeration. The particles are formed with small size less than 100 nm. Beside the effect of tripolyphosphate (TPP) as crosslinking agent which stabilize the formed chitosan nanocomposite. In addition, Tween 80 plays an extra role to disperse the formed nanoparticles and kept the particles from agglomeration or collision.

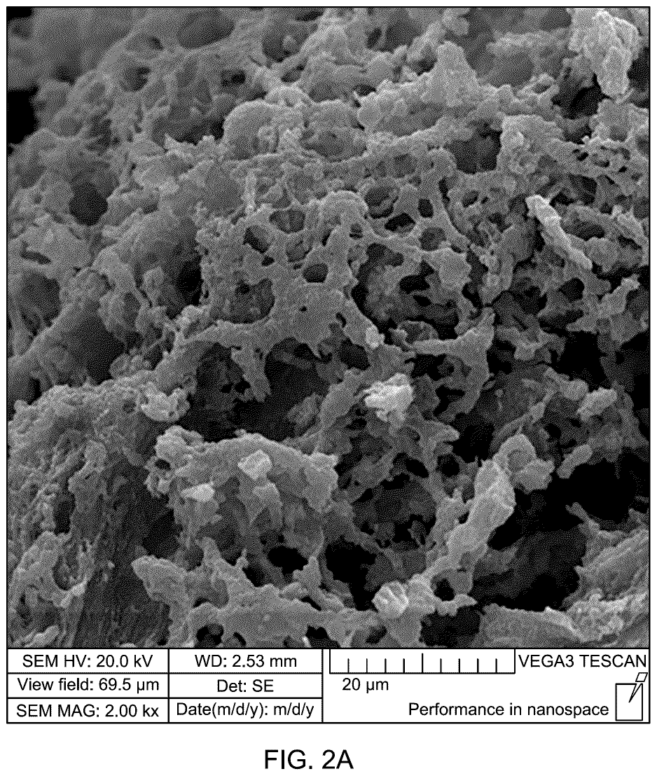

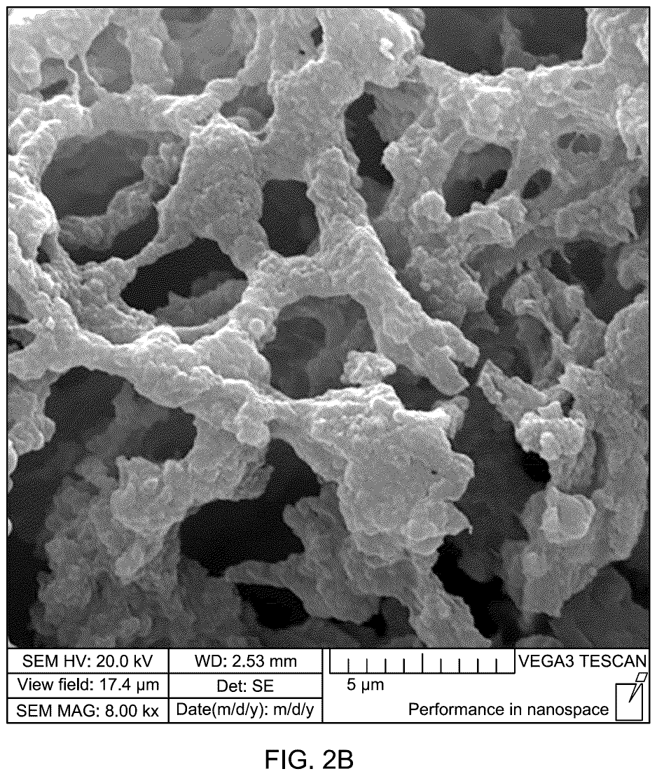

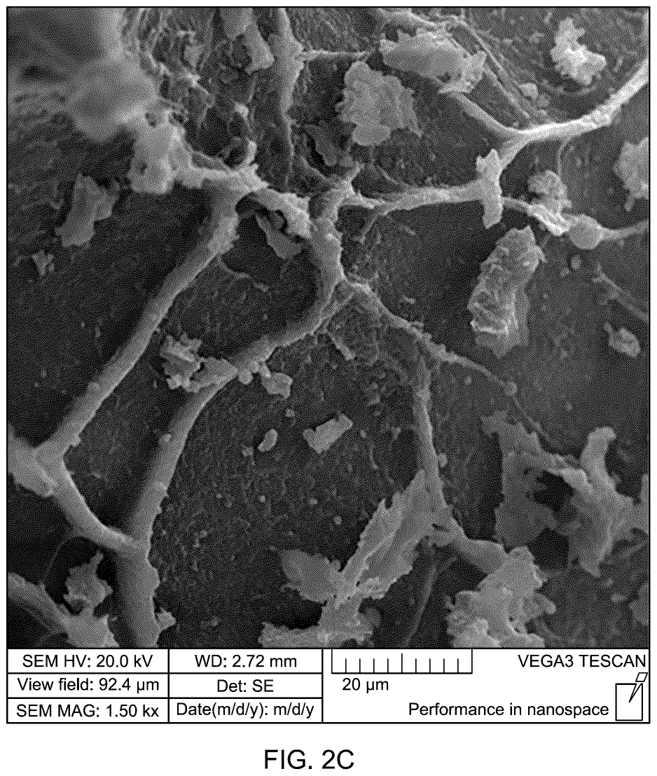

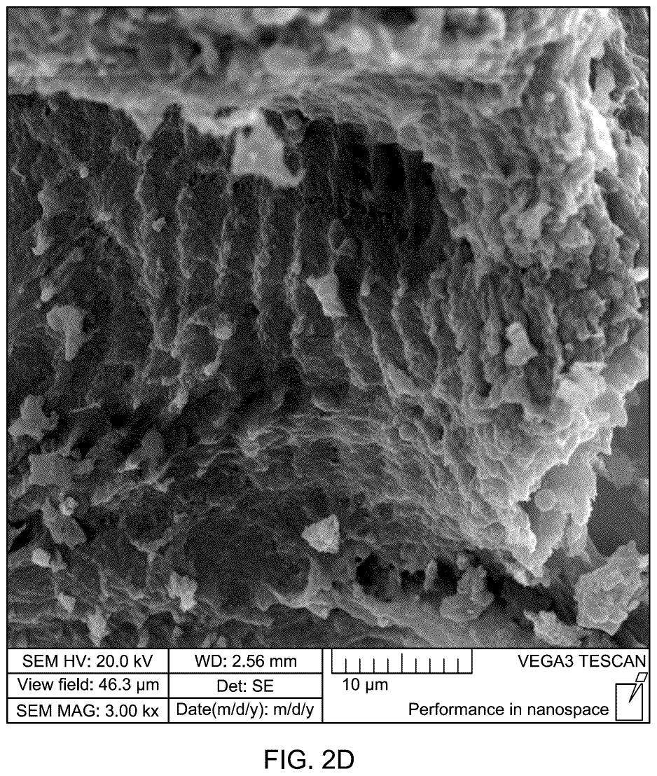

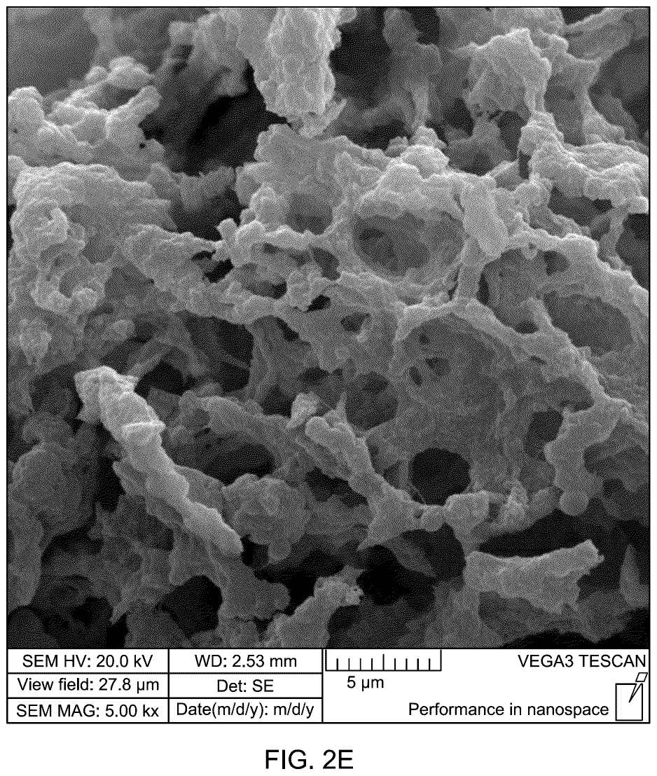

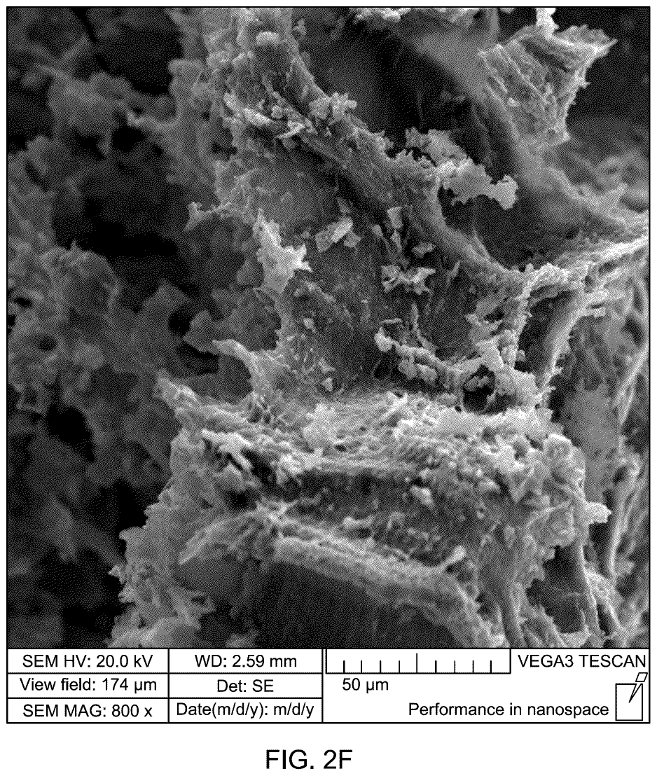

FIGS. 2A-2C shows the morphological and surface structure of GC@CsNC. The sample (GC@CsNC) was photographed at different magnifications. The different magnifications, especially the high magnification ones, are due to observing the small particles of GC that have been encapsulated inside the prepared chitosan nanoparticles. The formation of chitosan nanoparticles as an interconnected network was detected as a result of the interaction of chitosan with STPP. The small size of GE particles was confirmed, which formed in the form of small spherical particles. TEM was used to demonstrate the particle shape of the formed nanoparticles after dissolution in water. Thus, the particles were dispersed in water to form small particles. On the other hand, SEM was used to examine the morphological surface of the formed nanoparticles in a powder form. Thus, as detected from SEM, small particles of GC were adsorbed on the surface of CsNC. There are two different techniques, one of them was used to examine the particle shape and average size (TEM), while the other was utilized to examine the morphological surface (SEM). Additionally, the analysis was performed utilizing different states (TEM, colloidal solution) and (SEM Powder form).

Results of an EDX (Energy Dispersive X-ray) analysis of the GC@CsNC composition are tabulated in Table 1 below providing relative amounts of elements including C, O, Na, N, P, K, Cu, Mg, Zn, Ca, and Fe. The EDX data show that the detected elements are uniformly distributed through the GC@CsNC composition.

| | | | | | | Abs. | Rel. |
|---|---|---|---|---|---|---|---|
| | At. | | | Mass | | error (%) | error (%) |
| Element | No. | Netto | Mass % | Norm. % | Atom % | 1 sigma | 1 sigma |
| C | 6 | 2360 | 5.73 | 44.99 | 54.78 | 1.07 | 18.67 |
| O | 8 | 2074 | 4.72 | 37.10 | 33.91 | 0.91 | 19.31 |
| Na | 11 | 476 | 0.65 | 5.10 | 3.24 | 0.09 | 13.28 |
| N | 7 | 88 | 0.56 | 4.40 | 4.59 | 0.32 | 57.22 |
| P | 15 | 5409 | 0.55 | 4.31 | 2.04 | 0.05 | 8.84 |
| K | 19 | 254 | 0.23 | 1.83 | 0.69 | 0.04 | 18.30 |
| Cu | 29 | 46 | 0.16 | 1.26 | 0.29 | 0.05 | 31.19 |
| Mg | 12 | 66 | 0.07 | 0.54 | 0.32 | 0.04 | 52.38 |
| Zn | 30 | 9 | 0.03 | 0.22 | 0.05 | 0.01 | 34.73 |
| Ca | 20 | 15 | 0.02 | 0.16 | 0.06 | 0.01 | 26.68 |
| Fe | 26 | 4 | 0.01 | 0.09 | 0.02 | 0.01 | 50.73 |
| | | Sum | 12.73 | 100.00 | 100.00 | | |

### Colon Cancer Therapy

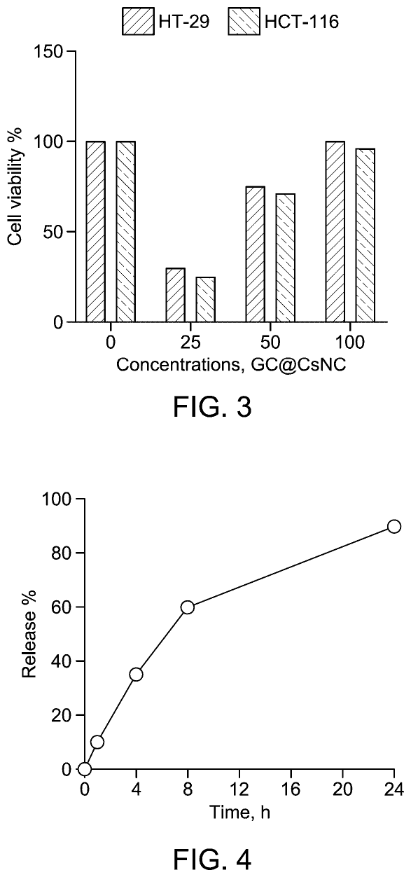

The results in FIG. 3 of the MTT assay demonstrates the dose-dependent cytotoxic effects of gingerol encapsulated in chitosan nanoparticles (GC@CsNC) on HT-29 and HCT-116 colon cancer cell lines. At lower concentrations (25 μg/mL), GC@CsNC exhibited significant cytotoxicity, reducing cell viability to 30% in HT-29 cells and 25% in HCT-116 cells. However, as the concentration increased (50 μg/mL-100 μg/mL), cell viability rose, suggesting reduced efficacy at higher doses, potentially due to saturation effects or altered cellular responses. The findings highlight the potential of GC@CsNC as an anticancer agent, particularly at optimized lower concentrations. The concentration of GC@CsNC (gingerol encapsulated in chitosan nanoparticles) in μg/mL represents the total amount of nanoparticles added to the cancer cell culture medium during the MTT assay.

### Kinetics Release

The release kinetics of gingerol from chitosan nanoparticles (CS) demonstrate a controlled and sustained release profile. Initially, there was no release observed at 0 hr, followed by a gradual release of 10% within the first hour, indicating the stability of the encapsulated gingerol. Over the next 4 h, 35% of gingerol was released, and by 8 h, 60% was released. The sustained release pattern highlights the efficacy of chitosan nanoparticles in providing a controlled delivery system, ensuring prolonged therapeutic availability of gingerol, which is beneficial for reducing dosing frequency and maintaining consistent drug concentrations in targeted applications, as shown in FIG. 4.

The kinetics release study of gingerol from chitosan nanoparticles (GC@CsNC) was conducted to evaluate the controlled and sustained release behavior under physiological conditions. The concentration of GC@CsNC used in this study was 5 mg/mL, ensuring an optimal balance between drug loading efficiency and controlled release characteristics. The release experiment was performed using the dialysis bag technique, where 2 mL of the GC@CsNC formulation was placed inside a dialysis membrane (12,000 MW cut-off), acting as the donor compartment. The dialysis bag was then immersed in a 50 mL phosphate-buffered saline (PBS) solution containing 0.5% sodium dodecyl sulfate (SDS) to maintain sink conditions, preventing drug precipitation and ensuring continuous diffusion. The system was maintained at 37° C. with constant stirring at 300 rpm to simulate physiological conditions, and the release medium was adjusted to pH value 7.2, representing various biological environments. At predetermined time points over 0-24 h, 1 mL of the release medium was withdrawn and replaced with fresh PBS-SDS solution to maintain consistent sink conditions. The concentration of released gingerol was measured using UV-visible spectrophotometry at 280 nm.

### Antibacterial Potential of GC@CsNC Against Some Nosocomial Pathogens

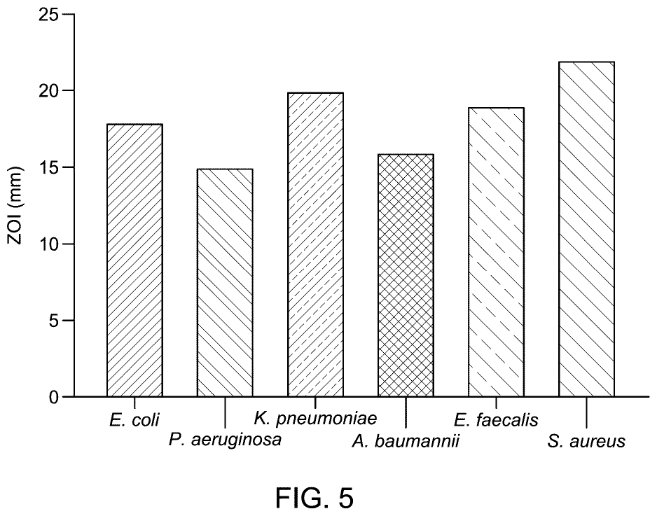

Results in FIG. 5 indicate that the GC@CsNC exhibited a robust antibacterial action against six pathogenic bacteria, each exhibiting varying degrees of susceptibility. The microorganisms exhibiting the greatest susceptibility were S. aureus (22 mm ZOI), K. pneumoniae (20 mm ZOI), and E. faecalis (19 mm ZOI). P. aeruginosa had the least sensitivity with a ZOI of 15 mm, while moderate activity was shown against E. coli (18 mm) and A. baumannii (16 mm). The findings demonstrate that GC@CsNC might be a useful wide-ranging antibacterial; and is effective optimally against tested bacterial species.

In the present disclosure, a sustainable way to administer medication that targets colon cancer and bacterial infections by encapsulating the bioactive molecule gingerol in biodegradable chitosan nanoparticles is described. The nanocomposite lowers systemic toxicity while improving stability, bioavailability, and therapeutic effectiveness of gingerol via targeted distribution and controlled release. GC@CsNC significantly reduced cell viability to 30% in HT-29 and 25% in HCT-116 at lower doses (25 μg/mL), indicating cytotoxicity against colon cancer cells.

Additionally, 60% of the gingerol was released continuously over the course of eight hours, indicating positive benefits. By preventing the growth of nosocomial pathogens including Klebsiella pneumoniae and Staphylococcus aureus by up to 22 mm, the nanocomposite showed strong antibacterial properties. This disclosure addresses two problems with traditional therapies such as antibiotics and chemotherapy: medication resistance and environmental harm. It does by using green chemical concepts and renewable materials. Through its use in cancer, antimicrobial treatment, and nutraceuticals, it has the potential to revolutionize sustainable medicine. In some embodiments, it integrates the potent therapeutic properties of gingerol with an advanced, biocompatible drug delivery system that involves encapsulating gingerol into chitosan nanoparticles. This advancement enhances stability and regulated release of gingerol, addressing three significant challenges in its therapeutic application: restricted solubility, inadequate bioavailability, and rapid degradation. Due to its favorable surface charge, the biodegradable and biocompatible biopolymer chitosan is an ideal carrier. It enables targeted delivery to diseased tissues, such as colon cancer cells, while sparing healthy ones. The process inhibits the rapid degradation of gingerol and facilitates its effective cellular uptake, hence avoiding any undesired side effects. The likelihood of accumulation over time is reduced since chitosan degrades into innocuous metabolites owing to its intrinsic biodegradability. This encapsulation method offers a secure and efficient dual-function treatment for bacterial infections and colon cancer.

Further, the nanocomposite adheres to the principles of green chemistry by using renewable and natural ingredients like chitosan and ginger. Ginger, a common agricultural crop, is responsibly harvested, while chitosan is made from chitin, a byproduct of the marine sector. Because this technique uses fewer synthetic chemicals and produces less waste, it lowers the harmful effects of production on the environment. The production and disposal of conventional pharmaceuticals often include harmful chemicals, leading to environmental damage. An emphasis on human and environmental health and sustainability is seen in using environmentally friendly processes throughout the production of this product. Further, the nanocomposite operates customized delivery approaches to lessen systemic toxicity. The chitosan nanoparticle are engineered to secrete gingerol in response to specific tumor microenvironment cues, such as acidic pH or enzymatic activity. This means gingerol directly reaches colon cancer cells, maximizing therapeutic efficacy while minimizing side effects, including gastrointestinal damage and immune suppression. By localizing gingerol's therapeutic effects to the cancer site, this individualized approach boosts the drug's anticancer effectiveness while improving patients' treatment experiences generally. Gingerol-encapsulated nanoparticles not only aid in cancer treatment but also eradicate a diverse array of infections, including drug-resistant strains. Gingerol's intrinsic antibacterial properties and the regulated release mechanism of chitosan nanoparticles effectively address cancer and bacterial infections in a singular, all-natural treatment. The flexibility of therapy facilitates improved healthcare solutions by reducing the need for individualized treatments.

In addition, gingerol has anti-inflammatory properties that might make it a potential choice for treating inflammatory disorders such as inflammatory bowel disease and arthritis. The formulation may potentially be used in medical treatment and nutraceuticals, which are dietary supplements or functional foods, to promote wellness and preventive healthcare.

Numerous modifications and variations of the present disclosure are possible in light of the above teachings. It is therefore to be understood that within the scope of the appended claims, the invention may be practiced otherwise than as specifically described herein.

1. A method of reducing cell viability of colon cancer cells, comprising:
contacting an encapsulated gingerol composition with the colon cancer cells at a concentration of 15 to 50 μg/mL,
wherein the encapsulated gingerol composition comprises gingerol nanoparticles extracted from Zingiber officinale, an ionic cross-linker, a polysorbate, and chitosan nanoparticles, wherein the ionic cross-linker is bonded to the chitosan,
wherein the encapsulated gingerol composition is in the form of nanoparticles having an average particle size of less than 100 nm,
wherein the contacting reduces the cell viability of the colon cancer cells to 40% or less, and
wherein 5-10% by weight of the gingerol nanoparticles in the encapsulated gingerol composition is released from the encapsulated gingerol composition within one hour of the contacting, 25-35% by weight of the gingerol nanoparticles in the encapsulated gingerol composition is released from the encapsulated gingerol composition within 4 hours, and 60% by weight of the gingerol nanoparticles in the encapsulated gingerol composition is released from the encapsulated gingerol composition within 8 hours.

2. The method of claim 1, wherein the colon cancer cells are HT-29 and the contacting at a concentration of 25 μg/mL reduces the colon cancer cell viability to 35% or less.

3. The method of claim 1, wherein the colon cancer is HCT116 and the contacting at a concentration of 15 μg/mL reduces the colon cancer cell viability to 30% or less.

4. The method of claim 1, wherein the ionic cross-linker is at least one selected from the group consisting of sodium tri metaphosphate (STMP), glutaraldehyde, genipin, epichlorohydrin, phosphoryl chloride, N,N′-methylenebisacrylamide, citric acid, adipic acid, starch dialdehyde, and sodium tripolyphosphate.

5. The method of claim 1, wherein the polysorbate is at least one selected from the group consisting of polysorbate 20, polysorbate 40, polysorbate 60, and polysorbate 80.

6. The method of claim 1, wherein the contacting comprises contacting the encapsulated gingerol composition with the colon cancer cells at a concentration of 15 to 50 μg/mL.

7. The method of claim 1, wherein the polysorbate is polysorbate 80.

8. The method of claim 1, wherein the ionic cross-linker is sodium tripolyphosphate.

9. A method of treating a bacterial infection, comprising:
contacting bacteria of the bacterial infection with an encapsulated gingerol composition at a concentration of 15 to 50 μg/mL to kill the bacteria,
wherein the encapsulated gingerol composition comprises gingerol nanoparticles extracted from Zingiber officinale, an ionic crosslinker, a polysorbate, and chitosan nanoparticles, wherein the ionic cross-linker is bonded to the chitosan,
wherein the encapsulated gingerol composition is in the form of nanoparticles having an average particle size of less than 100 nm, and
wherein the bacterial infection comprises at least one bacterium selected from the group consisting of Staphylococcus aureus, K. pneumoniae, E. faecalis, P. aeruginosa, E. coli, and A. baumannii.

10. The method of claim 9, wherein contacting the encapsulated gingerol composition with the bacteria forms a zone of inhibition of at least 20 mm against a Staphylococcus aureus bacterium.

11. The method of claim 9, wherein contacting the encapsulated gingerol composition with the bacteria forms a zone of inhibition of at least 18 mm against a Klebsiella pneumoniae bacterium.

12. The method of claim 9, wherein contacting the encapsulated gingerol composition with the bacteria forms a zone of inhibition of at least 17 mm against an Enterococcus faecalis bacterium.

13. The method of claim 9, wherein contacting the encapsulated gingerol composition with the bacteria forms a zone of inhibition of at least 13 mm against a Pseudomonas aeruginosa bacterium.

14. The method of claim 9, wherein contacting the encapsulated gingerol composition with the bacteria forms a zone of inhibition of at least 16 mm against an Escherichia coli bacterium.

15. The method of claim 9, wherein contacting the encapsulated gingerol composition with the bacteria forms a zone of inhibition of at least 14 mm against an Acinetobacter baumannii bacterium.

16. The method of claim 9, wherein the ionic cross-linker is at least one selected from the group consisting of sodium tri metaphosphate (STMP), glutaraldehyde, genipin, epichlorohydrin, phosphoryl chloride, N,N′-methylenebisacrylamide, citric acid, adipic acid, starch dialdehyde, and sodium tripolyphosphate.

17. The method of claim 9, wherein the polysorbate is at least one selected from the group consisting of polysorbate 20, polysorbate 40, polysorbate 60, and polysorbate 80.

18. The method of claim 9, wherein the ionic cross-linker is sodium tripolyphosphate.

19. The method of claim 9, wherein the polysorbate is polysorbate 80.

20. The method of claim 9, wherein the contacting comprises contacting the encapsulated gingerol composition with bacteria at a concentration of 15 to 50 μg/mL.
